(19) United States
(12) Patent Application Publication
(10) Pub. No.: US 2015/0174173 A1
(43) Pub. Date: Jun. 25, 2015

(54) STEM CELL TREATMENT FOR RADIATION EXPOSURE

(71) Applicant: University of Louisville Research Foundation, Inc., Louisville, KY (US)

(72) Inventors: Mariusz Z. Ratajczak, Louisville, KY (US); Janina Ratajczak, Louisville, KY (US); Magdalena Kucia, Louisville, KY (US); Denis Rodgerson, Malibu, CA (US)

(21) Appl. No.: 14/409,507

(22) PCT Filed: Jun. 24, 2013
(86) PCT No.: PCT/US13/47435

§ 371 Date: Dec. 19, 2014

Related U.S. Application Data
(60) Provisional application No. 61/663,600, filed on Jun. 24, 2012.

(51) Int. Cl. A61K 35/48

(52) U.S. Cl. CPC A61K 35/48

(57) ABSTRACT

The invention provides adult pluripotent stem cells (PSC) for treatment or prophylaxis for radiological exposure. In an embodiment of the invention, the cells are very small embryonic like stem cells (VSELs). The VSELs can be used to rescue the hematopoietic and immune systems of individuals suffering from the delayed effects of acute radiation syndrome (ARS).

### CROSS REFERENCE TO RELATED APPLICATIONS

This application is a United States National Stage application filed under 35 U.S.C. §371 of PCT International Patent Application Serial No. PCT/US2013/047435, filed Jun. 24, 2013, which itself is based on and claims priority to U.S. Application No. 61/663,600, filed Jun. 24, 2012. The disclosure of each of these applications is incorporated herein by reference in its entirety.

This invention was made with government support under grant R43AI098325 awarded by the National Institutes of Health. The government has certain rights in the invention.

### FIELD OF THE INVENTION

The invention provides adult pluripotent stem cells (PSC) for treatment or prophylaxis for radiological exposure. In an embodiment of the invention, the cells are very small embryonic like stem cells (VSELs). The VSELs can be used to rescue or reestablish the hematopoietic and immune systems of individuals suffering from the delayed effects of radiation exposure, such as acute radiation syndrome (ARS).

### BACKGROUND OF THE INVENTION

Acute Radiation Syndrome (ARS) is a combination of clinical symptoms that occurs in stages. Hematopoietic syndrome arises from the depletion of parenchymal stem cells with consequential bone marrow (BM) failure after exposure to lethal radiation. BM cells are the building blocks for red and white blood cells, and platelets. Consequently, infection-fighting cells and antibody production are impaired, and clotting mechanisms become less effective. Death can occur within 6 weeks following radiation exposure. The primary cause of death from radiation injury is infection that is unrestrained due to the failure of the immune system and the inability of the bone marrow to produce infection fighting cells.

In the event of a nuclear accident or terrorist bomb, large numbers of casualties will have been exposed to acute high-dose radiation. Those exposed to even low levels of radiation will have compromised immune systems such that the virulence and infectivity of biological agents is dramatically increased. A compromised immune system exacerbates the effects of infectious agents and may preclude use of vaccines.

The dose of radiation and resulting disorders depend upon several factors, including the source, type of ionizing radiation, absorbed dose, proximity to the source, weather, and individual sensitivity. The U.S. occupational annual limit is 0.05 Gray (Gy) and the lethal dose (LD_{50/60}) where 50% of individuals will die within 60 days without medical intervention is 3.5-4.5 Gray. In general, acute radiation syndrome does not develop when exposure is <1 Gy. Individuals exposed to radiation in the range of 1.0 to 8.0 Gy develop symptoms that reflect injury to the hematopoietic (bone marrow) system. With radiation exposure of 6.0 to 8.0 Gy, a gastrointestinal syndrome develops which is superimposed on the hematopoietic syndrome. Supportive and comfort care is usually indicated for individuals exposed to >10 Gy, since their prognosis is grave. Individuals receiving such high doses are usually killed or severely injured by the blast and thermal effects of a nuclear detonation, although doses in this range could result from accidental or deliberate exposure within a reactor facility or fuel reprocessing plant.

Stem cell transplantation is the only intervention that can save a fatally irradiated person. The cure rate for this treatment can be high, provided the treatment is delivered within 7-10 days following exposure to radiation. Stem cell transplantation was used successfully following the Tokaimura nuclear reactor accident in Japan. Stem cell transplantation administered after the Chernobyl accident was less successful because of long delays in initiating treatment. More than 30 stem cell transplantations have been carried out for radiation accident victims. All of these transplants have been allogeneic and most have been unsuccessful because of rejection and graft-versus-host disease that occurs as an unwanted consequence of immune reconstitution with foreign cells. Consequently, development of autologous stem cell therapies that are not rejected could greatly increase the success of treating radiation poisoning.

There are limited ways to acquire autologous human stem cells to generate new hematopoietic stem cells and rescue the immune system of irradiated individuals. One approach could involve administering G-CSF to irradiated patients to mobilize resident CD34⁺ stem cells. The problem with this approach is that hematopoietic stem cells are highly sensitive to ionizing radiation and as such an individual's ability to mobilize competent hematopoietic stem cells following exposure to radiation will be severely limited. This fact essentially eliminates the possibility of post-exposure harvesting of hematopoietic stem cells for autologous therapy.

Until now, the treatment of radiation sickness by stem cell therapy has been based on the same process as bone marrow transplantation following myeloablation for hematological malignancies. The therapy has been aimed at reconstituting the immune system through the engraftment of healthy hematopoietic stem cells (for which CD34 positive cells are surrogates), and has offered a means of restoring the compromised immune system, thereby eliminating the basis for infection, hemorrhage, etc. Timely bone marrow recovery is key to survival. Hematopoietic growth factors stimulate proliferation of granulocyte precursors, but if stem cells are eradicated, bone marrow aplasia will ensue. A small number of radiation accident victims have undergone allogeneic transplantation from a variety of donors in an attempt to overcome radiation-induced aplasia.

Human adult pluripotent stem cells are a sub-population of CD34+ cells that can be collected from mobilized peripheral blood by apheresis. These are small cells (<7 microns) that have many of the characteristics of embryonic stem cells, but are not tumor forming. These cells have been called very small embryonic-like stem cells (VSEL). Significant quantities of VSELs can be obtained from the peripheral blood of humans. Results show that after G-CSF mobilization, human peripheral blood contains a population of lin⁻CD45⁻ mononuclear cells that express CXCR4, CD34, and CD133. These CXCR4⁺ CD133⁺ CD34⁺ lin⁻ CD45⁻ cells are highly enriched for mRNA for intra-nuclear pluripotent embryonic transcription factors such as Oct-4 and Nanog, and also express the cell surface marker SSEA-4. VSELs can differentiate into multiple cell types in vitro and in mice. Human VSELs have been shown to form osteocytes, adipocytes and endothelial cells in an animal model and to support angiogenesis. Under suitable conditions, VSELs can differentiate into hematopoietic repopulating cells, and type 2 pneumocytes, and have shown to be mobilized into the peripheral blood of humans following myocardial infarction, stroke and burns.

### SUMMARY OF THE INVENTION

The bone marrow contains a heterogeneous population of more primitive uncommitted stem cells that have several, or all of, the cardinal properties of pluripotent stem cells. These pluripotent stem cells have the potential to differentiate into all three germ layers and hence regenerate not only hematopoietic, but all tissues. According to the invention, the use of pluripotent VSEL stem cells to treat radiation exposure, including ARS, offers benefits of allogeneic HSCs without the disadvantages associated with allogeneic bone marrow transplantation, and also offers a regenerative foundation for cells of the gastrointestinal tract, nervous tissue and others.

Significant quantities of very small embryonic-like stem cells (VSELs) can be obtained from the peripheral blood of humans following mobilization with G-CSF. Results show that after G-CSF mobilization, human peripheral blood contains a population of lin⁻CD45⁻ mononuclear cells that express CXCR4, CD34, and/or CD133. These CXCR4⁺ CD133⁺ CD34⁺ lin CD45⁻ cells are highly enriched for mRNA for intra-nuclear pluripotent embryonic transcription factors such as Oct-4 and Nanog, and also express the cell surface marker SSEA-4, the early embryonic glycolipid antigen commonly used as a marker for undifferentiated pluripotent human embryonic stem cells. VSELs can differentiate into multiple cell types. Before G-CSF mobilization, very few VSELs are detectable in peripheral blood; following mobilization, there is a very significant increase with in excess of 106 VSELs present in the apheresis product, representing much less than 0.0001% of total nucleated cells.

Moreover, VSELs are highly resistant to radiation damage as compared to a general population of hematopoietic stem cells. VSELs can not only tolerate radiation doses in excess of 1 Gy with retention of their ex vivo pluripotent differentiating ability, but appear to be induced into proliferation by the radiation. VSELs can be differentiated to hemato/lymphopoietic lineage, and can rescue the immune system of subjects exposed to lethal radiation. Also important is that the ex vivo expansion of VSELs when it is necessary, requires only 5-10 days in culture. The importance of an autologous source for hematopoietic/lymphopoietic rescue cannot be overstated.

According to the invention, VSELs are used to rescue the immune system of individuals suffering from the delayed effects of acute radiation syndrome (ARS). In certain embodiments, the VSELs are autologous. In other embodiments, the VSELs are allogeneic. The VSELs may be administered to a subject without rejection or induction of graft versus host disease. The cells are pluripotent and can be expanded and differentiated to all three germ cell lineages. The VSELs can be differentiated to hemato/lymphopoietic lineage and restore their functions. VSELs also bring about regeneration of other tissue damaged by radiation, such as gut, lung, etc.

In certain such embodiments, the VSELs are collected from an irradiated subject. The invention provides a method of treating radiation exposure in a subject, which comprises collecting VSELs from the irradiated subject (i.e., collecting VSELs after the radiation exposure), and administering an effective amount of VSEL stem cells to treat the radiation exposure. In an embodiment of the invention, the subject is administered an agent to mobilize VSELs prior to collection. The collected VSELs may be expanded and/or directed or selected to differentiate prior to administration to the subject. In one embodiment, an expanded population of VSEL stem cell-derived cells capable of differentiation into hematopoietic/lymphopoietic stem cells is produced and administered to the subject. In certain embodiments, a radiation exposure victim is treated with autologous VSELs and/or VSEL-derived cells. In certain embodiments, a radiation exposure victim is treated with allogeneic VSELs and/or VSEL-derived cells. As used herein, VSEL-derived cells include cells obtained from VSELs by expansion, and cells expressing most, if not all, VSEL markers (for example, markers of pluripotent stem cells) and also one or more markers indicative of differentiation towards a particular or selected cell type. For example, growing hVSELs in serum-free medium with SCF, TPO, and Flt3-L increases expression of the hematopoietic marker CD45.

The invention also provides for pre-exposure collection of autologous stem cells for example, from high-risk individuals before they are exposed to radiation. Pre-exposure collection and transplantation of autologous VSELs has several advantages: First, transplantation does not cause graft versus host disease (GVHD), which further exacerbates injuries (e.g., gut injury) mediated by radiation exposure. Second, it does not require immunosuppressants, which make radiation victims more susceptible to severe infections. Third, VSELs can induce more rapid hematopoietic recovery than can hematopoietic growth-factor support alone or bone-marrow cells. Fourth, VSELs are easy to store by cryopreservation. Fifth, the short-term and long-term safety of peripheral blood stem cell collection has been confirmed in a large number of healthy donors for patients with hematological cancers. As radiation is a well known carcinogen in the long term, VSEL collection and banking is also beneficial for treatment of leukemias that would be expected to arise over time. The invention also provides for administration of allogeneic VSEL stem cells and VSEL-derived cells.

The invention also provides for collection and administration of autologous human VSELs to subjects prior to, or during the course of, therapies that reduce or ablate the subject's hematopoietic/lymphopoietic system, such as, for example, treatment for cancer with radiation or chemotherapy. Chemotherapy and or radiotherapy can impair erythropoiesis as a result of a direct myelotoxic effect on the bone marrow red blood cell (RBC) progenitors. In contrast, VSELs are quiescent and resistant to agents that primarily target cycling cells. Further, certain subjects may be insufficiently sensitive to erythropoiesis-stimulating agents (ESAs) such as erythropoietin (EPO). According to the invention, subjects treated with radiotherapy or chemotherapy benefit from collection of VSELs and administration of expanded and/or primed VSELs.

### DESCRIPTION OF THE FIGURES

FIG. 1—Properties of hVSELs following isolation from peripheral blood by apheresis. Expression of the pluripotency markers Oct-4 and Nanog detected by RT-PCR in hVSEL compared with expression in total nucleated cells isolated blood.

FIG. 2—Expansion of hVSELs using MSC feeder cells. Human BM-derived MSCs (2000/well) were seeded on 96-well plates as a feeder layer. 500 VSELs were plated onto the MSCs and cultured under serum- and xeno-free conditions with SCF, FLT-3 and TPO for 12 days.

FIG. 3—Differentiation of Human VSELs to hematopoietic lineage. Culture of hVSELs in Serum-free medium with SCF, TPO, and Flt3L increases expression of the hematopoietic marker CD45.

FIG. 4—Formation of hemangioblasts with hematopoietic potential. CFU-M: colony forming unit-macrophage; CFU-G: colony forming unit-granulocyte; BFU-E: burst forming unit-erythrocyte.

FIG. 5—Clonogenic potential in vitro of hVSELs. FACS-sorted hVSELs from umbilical cord blood were plated for 5 days over OP9 stromal cells and subsequently tested for clonogenic potential in MethoCult cultures. The clonogenic potential of cells derived from OP9-primed hVSELs (CD45⁻CD133⁺ALDH^{low} and CD45⁻CD133⁺ALDH^{high}) increases gradually after replating into secondary and tertiary cultures.

FIG. 6—Strategy for reconstituting lethally irradiated C57Bl/6 mice by transplantation of expanded GFP-marked VSELs from lethally irradiated donors. Either 1×10⁵ or 2×10⁵ GFP⁺ donor cells that had been expanded in co-culture with OP9 cells were transplanted by intravenous injection.

FIG. 7—Chimerism of lethally irradiated mice reconstituted with GFP-marked VSELs. The proportion of GFP⁺ cells in bone marrow, spleen, and peripheral blood in recipient mice, two weeks and 2 months after transplantation, is depicted.

### DETAILED DESCRIPTION OF THE INVENTION

It has been observed that VSELs are resistant to lethal irradiation, which destroys hematopoietic stem cells and most other proliferating cells in the body. In subjects exposed to lethal radiation, VSELs remain alive in bone marrow (BM) and appear to proliferate in response to the tissue damage caused by irradiation.

According to the invention, autologous human VSELs are isolated from a subject and used to treat radiation exposure, such as acute radiation sickness (ARS). The VSELs can be isolated from bone marrow, and are readily mobilized and collected from peripheral blood. When available, VSELs may also be isolated from umbilical cord blood. In certain embodiments of the invention, VSELs are isolated after a subject has been exposed to radiation, and administered back to the subject to treat the exposure. After collection and prior to administration, the VSELs may be expanded and/or induced to differentiate toward one or more selected cell types. The VSELs may optionally be stored in in preparation for administration over time, such as over a period of days, weeks, months, or years. According to the invention, mobilization, expansion, and differentiation procedures may be selected to optimize the number and type of cells administered and minimize the time between radiation exposure and VSEL administration. In certain embodiments, heterologous human VSELs are isolated from a first subject, optionally expanded and/or induced to differentiate toward one or more selected cell types, and administered to a second subject to treat a radiation exposure. In such embodiments, the first subject can also have suffered radiation exposure prior to VSEL donation.

According to the invention, autologous human VSELs can be isolated from a subject expected or threatened to be exposed to, hazardous radiation. After collection and prior to administration, the VSELs may be expanded and/or induced to differentiate toward one or more selected cell types. Optionally, the collected VSELs may be stored. According to the invention, mobilization, expansion, and differentiation procedures may be selected to optimize the number and type of cells administered. In certain embodiments, human VSELs are isolated before the radiation exposure, optionally stored, expanded, and/or differentiated, and administered to the subject after the radiation exposure. In certain embodiments, isolated (and optionally stored, expanded, and/or differentiated) VSELs are administered more than once to the subject, over a period of days, weeks, or months. Such multiple administrations may involve repeated or continuous expansion and/or differentiation of collected or stored VSELs. Also according to the invention, heterologous human VSELs are isolated from a first subject, optionally stored, expanded and/or induced to differentiate toward one or more selected cell types, for administration to a second subject expected or threatened to be exposed to, hazardous radiation.

In other embodiments, isolated (and optionally stored, expanded, and/or differentiated) VSELs are prophylactically administered to a subject prior to, or concurrent with, a radiation exposure. In such embodiments, VSELs may be administered more than once to the subject, over a period of days, weeks, months, or years. Similarly, in certain embodiments, heterologous human VSELs are isolated from a first subject, optionally stored, expanded and/or induced to differentiate toward one or more selected cell types, and administered to a second subject prior to or concurrent with a radiation exposure.

According to the invention, radiation is ionizing radiation, including, but not limited to, beta radiation, gamma radiation, X-rays, cosmic radiation, and solar particle event radiation. Occupational exposure to ionizing radiation can occur in a range of industries, including, without limitation, mining and milling, in medical institutions, in educational and research establishments, in nuclear fuel cycle facilities, and during spaceflight.

The term “very small embryonic-like stem cell” is also referred to herein as “VSEL stem cell” or “VSEL” and refers to certain stem cells that are pluripotent. In certain embodiments, the VSEL stem cells (“VSELs”) are human VSELs and may be characterized as lin⁻, CD45⁻, and CD34⁺. In certain embodiments, the VSELs are human VSELs and may be characterized as lin⁻, CD45⁻, and CD133⁺. In certain embodiments, the VSELs are human VSELs and may be characterized as lin⁻, CD45⁻, CD133⁺ and CD34⁺. In certain embodiments, the VSELs are human VSELs and may be characterized as lin⁻, CD45⁻, and CXCR4⁺. In some such embodiments, the VSELs are lin⁻, CD45⁻, CXCR4⁺, and CD34⁺. In other such embodiments, the VSELs are lin⁻, CD45⁻, CXCR4⁺, and CD34⁻. In certain embodiments, the VSELs are human VSELs and may be characterized as lin⁻, CD45⁻, CXCR4⁺, CD133⁺, and CD34⁺. In certain embodiments, human VSELs express at least one of SSEA-4, Oct-4, Rex-1, and Nanog. VSELs may also be characterized as possessing large nuclei surrounded by a narrow rim of cytoplasm, and containing embryonic-type unorganized chromatin. In some embodiments, VSELs have high telomerase activity. In certain embodiments, human VSELs may be characterized as lin⁻, CD45⁻, CXCR4⁺, CD133⁺, Oct 4⁺, SSEA4⁺, and CD34⁺. In certain embodiments, the human VSELs may be less primitive and may be characterized as lin⁻, CD45⁻, CXCR4⁺, CD133⁻, and CD34⁺. In certain embodiments, the human VSELs may be enriched for pluripotent embryonic transcription factors, e.g., Oct-4, Sox2, and Nanog. In certain embodiments, the human VSELs may have a diameter of 4-5 μm, 4-6 μm, 4-7 μm, 5-6 μm, 5-8 μm, 6-9 μm, or 7-10 μm. VSELs administered according to the invention can be collected and enriched or purified and used directly, or frozen for later use. VSELs from cord blood have also been characterized as being CD133⁺/GlyA⁻/CD45. In some embodiments, the CD133⁺/GlyA⁻/CD45⁺ cells are ALDHhigh cells. In some embodiments, the CD133⁺/GlyA⁻/CD45⁺ cells are ALDH^{low} cells. (See, e.g., WO 2010/057110, entitled Methods And Compositions For Long Term Hematopoietic Repopulation).

The invention also features VSEL-derived cells, which are cells that are differentiating from VSELs along a hemato/lymphopoietic or other lineage. Such cells may express CD45 as they differentiate. VSEL-derived cells may be found in expanded VSEL cultures, and are expected to be present in vivo as administered VSELs differentiate. For example, clonogenic derivatives of cord blood VSELs include CD133⁺/GlyA⁻/CD45⁻ cells and CD133⁺/GlyA⁻/CD45⁺ cells, each of which can be ALDH^{high} or ALDH^{low}.

As used herein, the phrase “mobilizing agent” refers to a compound (e.g., a peptide, polypeptide, small molecule, or other agent) that when administered to a subject results in the mobilization of a VSEL stem cell or a derivative thereof from the bone marrow of the subject to the peripheral blood. Stated another way, administration of a mobilizing agent to a subject results in the presence in the subject's peripheral blood of an increased number of VSEL stem cells and/or VSEL stem cell derivatives than were present therein immediately prior to the administration of the mobilizing agent. It is understood, however, that the effect of the mobilizing agent need not be instantaneous, and typically involves a lag time during which the mobilizing agent acts on a tissue or cell type in the subject in order to produce its effect. Preferably, the one or more stem cell mobilizing agent is selected from the group consisting of G-CSF, GM-CSF, dexamethasone, a CXCR4 receptors inhibitor and a combination thereof. In some embodiments, the mobilizing agent comprises at least one of granulocyte-colony stimulating factor (G-CSF) and a CXCR4 antagonist (e.g., a T140 peptide; Tamamura et al. (1998) 253 Biochem Biophys Res Comm 877-882). Examples of CXCR4 inhibitors that have been found to increase the amount of VSELs in the peripheral blood include, but are not limited to, AMD3100, ALX40-4C, T22, T134, T140 and TAK-779. See also, U.S. Pat. No. 7,169,750, incorporated herein by reference in its entirety.

These stem cell potentiating agents may be administered to the person before the collecting step. For example, the potentiating agent may be administered at least one day, at least three days, or at least one week before the collecting step. Preferably, the potentiating agents are administered to a subject at least twice over a 2 to 6 day period. For example, the potentiating agent may be administered on day 1 and day 3 or may be administered on day 1, day 3, and day 5 or, alternatively, day 1, day 2, and day 5. Most preferably, the potentiating agents are administered to a subject twice for consecutive days over a 3 day course. Thus, according to a preferred embodiment, the potentiating agents are administered to a subject on day 1 and day 2, for example, from 12 to 36 hours apart, or from 18 to 30 hours apart, followed by collection by apheresis on day 3. Such a time course provides a high yield of VSELs in a reasonably short period of time.

Effective amounts of mobilizing agents are known in the art. For example, G CSF can be administered at a dose of about 10 to about 16 μg/kg/day or more. In certain embodiments, at least two doses of G-CSF of about 1 μg/kg/day to 8 μg/kg/day are administered. In certain embodiments, G-CSF is administered to a subject at a dose of about 4 to about 6 μg/kg/day or equivalent thereof. In certain embodiments, about 50 μg to about 800 μg per dose, or about 300 μg to about 500 μg per dose, of G-CSF is administered subcutaneously to the subject. In one exemplary embodiment, mobilization involves treating a subject with low dose (480 μg/day) G-CSF for several days to mobilize resident VSELs in BM to migrate to blood. G CSF is FDA approved and short-term treatment has been found to be safe in humans.

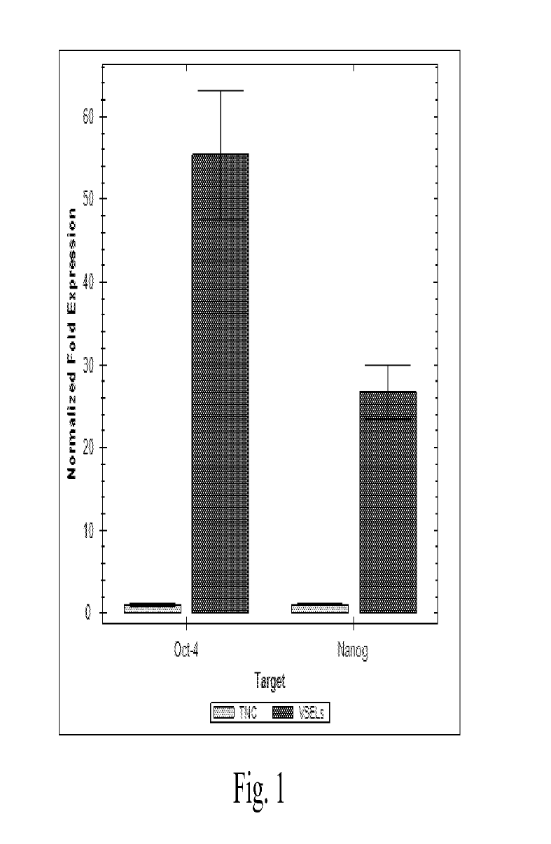

VSELs can be collected and purified by any method. In one embodiment, VSELs are isolated from the blood by apheresis and the product further size fractionated to collect cells measuring 5-7 μM in range. The cellular fraction is subjected to FACS isolation of CD45-cell population using a high speed flow sorting instrument. This process takes 3 hours. Studies from 8 healthy volunteers show an average of 16.9 million VSELs can be collected. FIG. 1 shows the isolated VSEL population expresses Oct-4 and Nanog mRNA identified by RT-PCR and expression of these embryonic markers were at far higher levels than seen in the total nucleated cells isolated in the blood samples. Enough VSELs to start regenerative therapy can be expanded and primed in about 24 hr following collection.

WO 2011/069117 describes a method of isolation of stem cell populations from peripheral blood using sized-based separation. Fresh apheresed cells are lysed with 1× BD Pharm Lyse Buffer, in a ratio of approximately 1:10 (vol/vol) to remove red blood cells. After washing, cells are counted, and 2-2.5×1010 total nucleated cells are loaded onto the ELUTRA® Cell Separation System (CaridianBCT) at a concentration of 1×10⁸ cells/ml. Cells are then collected in 900 ml PBS+0.5% HSA media in each bag at different flow rates. Typically, six fractions are collected with a centrifugation speed of 2400 rpm. Finally, cells from all fractions are transferred into tubes and spun down at 600×g for 15 minutes. Size characteristics of the fractions are confirmed by evaluating SSC and FSC. As disclosed therein, Fraction 2 (50 mL/min) is highly enriched in VSELs and can be used to provide populations of VSELs for clinical applications. The procedure can be adapted to other equipment. The populations may be further purified by FACS.

Using methods that separate cells based on size or density such as differential centrifugation, percoll gradient centrifugation, and counterflow centrifugal elutriation, it was observed that the Lin⁻CD45⁻CD34⁺133⁺ events fall into two separate populations with different physical characteristics—a major population (approximately 98% of Lin⁻CD45⁻CD34⁺133⁺ events) of objects that are very small (<4 μm), very light, and stain negatively or dimly with the nuclear dye DRAQ5, and a minor population that is larger (5-10 μm), heavier, and that stains brightly with DRAQ5. FACS sorting of the two populations followed by cytospin and diff-quick stain showed that the minor population consists of small nucleated cells, whereas the major population consists of membrane-bound objects that do not have a cell nucleus. By light microscopy and transmission electron microscopy these objects have the appearance of extracellular vesicles. Although most are roughly the size of platelets, their morphologic appearance is quite different from platelets. The two populations of Lin⁻CD45⁻CD34/133⁺ events are also found in umbilical cord blood, although at different frequencies than in mobilized adult blood (1 for every 5 hematopoietic progenitors, with 94% of the events being DRAQ5⁻). Accordingly, when VSELs are isolated or purified by flow cytometry, a nuclear marker such as DRAQ5 can be useful to quantify nucleated VSELs among other cell-like objects cells having VSEL markers or characteristics. As with the DRAQ5⁺ nucleated VSELs, the enucleated particles, which express markers of VSELs, may also be isolated and used in the invention.

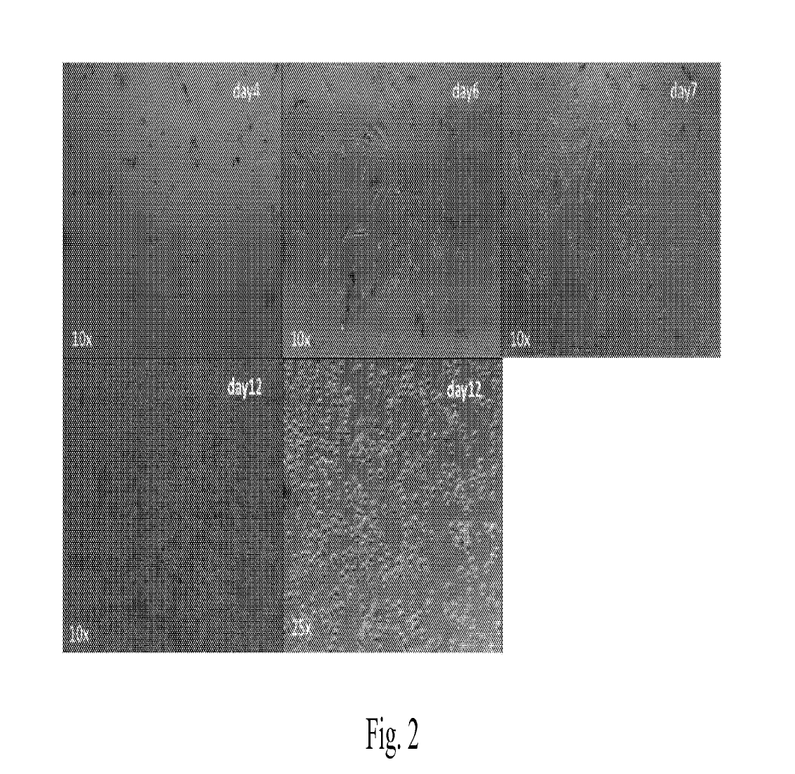

hVSELs can be expanded through multiple passages. For example, by growing hVSELs on a feeder layer of OP9 cells or bone marrow-derived MSCs, the number of VSELs and VSEL-derived stem cells can be increased in a short period of time. When cultured on a feeder layer of bone marrow-derived MSCs in serum-free medium in the presence of stem cell factor (SCF), FLT3 ligand (Flt3L), thrombopoietin (TPO) and basic Fibroblast Growth Factor (bFGF), a 200-fold expansion of hVSELs was achieved in a short time (FIG. 2), allowing enough hVSELs to be generated from a patient to provide cells for transplants and booster transplants.

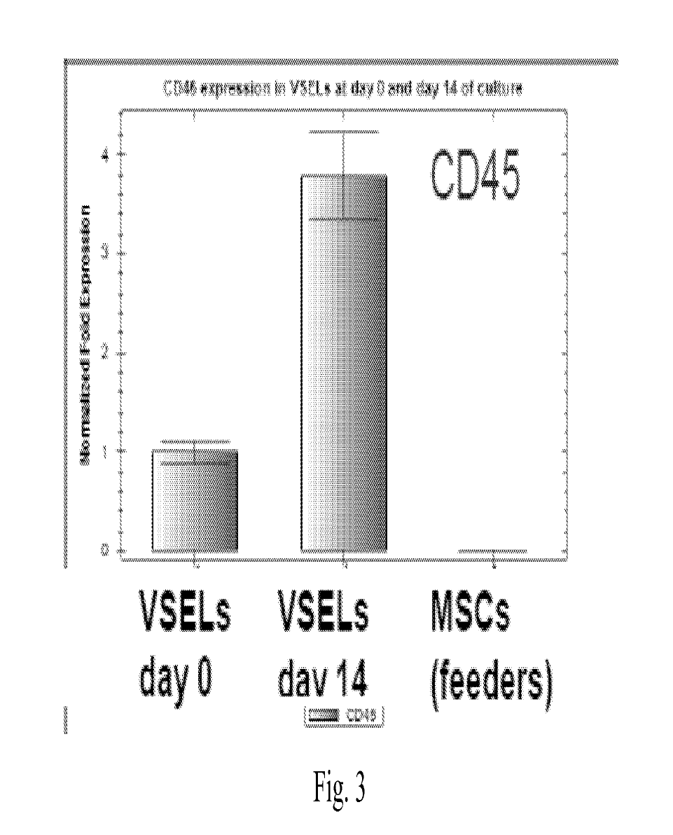

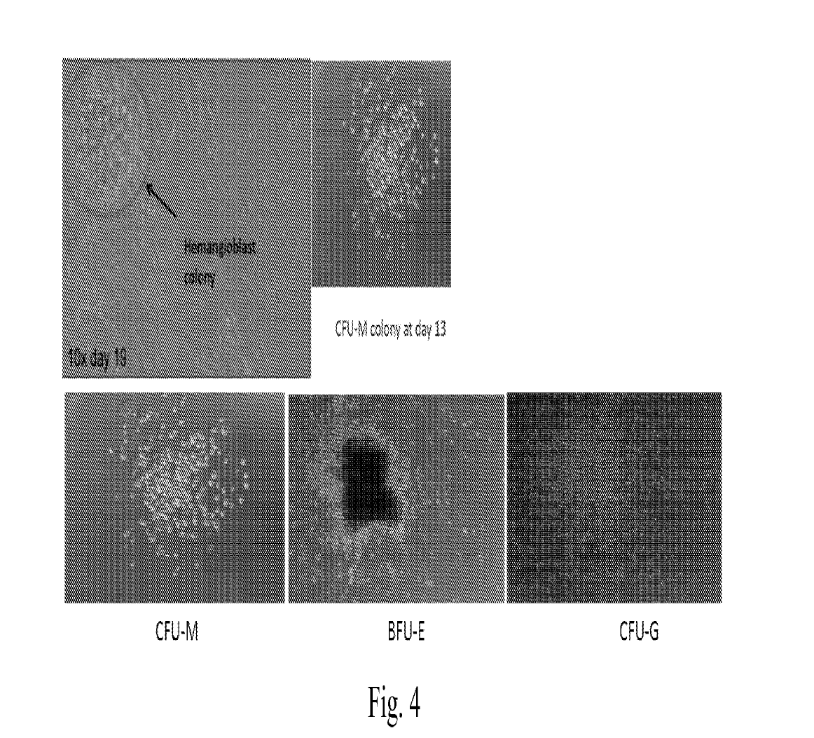

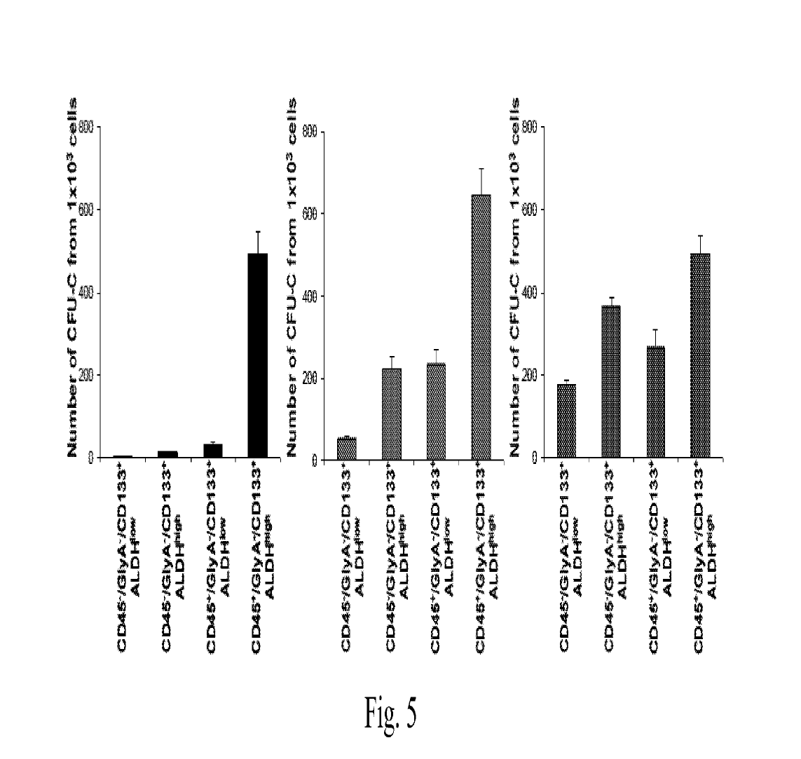

Further, VSELs can be primed to differentiate towards hematopoietic/lymphopoietic lineage. One way is priming by co-culture with OP9 cells. OP9 priming induces expression of hematopoietic genes (e.g., Ikaros, GATA-2, HoxB4, PU.1, c-myb) that are not expressed in freshly isolated VSELs. As the VSELs acquire expression of hematopoietic genes, they lose expression of Oct-4. Also, growing the VSELs in serum-free medium with SCF, TPO and Flt3-L also increases expression of the hematopoietic marker CD45 (FIG. 3, upper left). With time, hemangioblasts form which are progenitors of hematopoietic and endothelial cells (CD34+, CD133+, express Flk-1, mesodermal gene T (brachyury)) (FIG. 4, upper left). Hemangioblasts can be plated on methylcellulose to perform CFU-assays to test their hematopoietic potential. For example, colony forming unit-macrophage (CFU-M), colony forming unit-granulocyte (CFU-G) and Burst forming unit-erythrocyte (BFUE) can be observed (FIG. 4). The VSELs can be from marrow, blood, or umbilical cord blood (FIG. 5).

According to the invention, the time for which VSELs are expanded and/or primed before administration is selected according to the number of VSELs isolated from a donor, the number of VSELs desired to be administered, and the urgency for administering the cells. According to the invention, the VSELs can be co-cultured, for 1, 2, 3, 4, 5, 6, 7, 8, 9, or 10 days or longer. In certain embodiments, the VSELs are co-cultured with OP9 cells for 5-10 days.

Autologous VSEL cell collection and storage is desirable for select populations, such as those who, by the nature of their work are at high risk of exposure to ionizing radiation following a nuclear attack or accident. Examples include military and government staff who are specially trained to respond to nuclear or radiological incidents. Allogeneic VSELs can also be collected and stored for use in such populations.

In embodiments of the invention, freshly isolated VSELs and/or expanded and/or differentiated VSELs are administered. Alternatively, cryopreserved VSELs can also be employed. More particularly, once collected VSELs can be cryopreserved at any point prior to administration to a subject. Thus the VSELs may be cryopreserved after collection, expansion, and/or differentiation. Methods for cryopreserving VSELs for later processing and/or administration to a subject are known to one of ordinary skill in the art.

The presently disclosed subject matter provides methods for treating radiation exposure. In some embodiments, the methods comprise administering to the subject a composition comprising a plurality of isolated VSELs and/or VSEL-derived cells in a pharmaceutically acceptable carrier in an amount and via a route sufficient to allow at least a fraction of the administered cells to engraft and/or differentiate therein.

In some embodiments, the target site comprises the bone marrow of the subject. The invention provides a method comprising administering to a subject with at least partially absent bone marrow a pharmaceutical preparation comprising an effective amount of isolated VSELs and/or VSEL-derived cells, wherein the effective amount comprises an amount sufficient to engraft in the bone marrow of the subject. As used herein, the phrase “a subject with at least partially absent bone marrow” refers to a subject that has been accidentally or intentionally treated or irradiated, for example as a result of a radiation accident or a myeloablative or myeloreductive treatment, which eliminates at least a part of the bone marrow in the subject. Bone marrow transplantation is a technique that generally would be well known to one of ordinary skill in the art after review of the instant disclosure. Several U.S. and other patents and patent applications have been published which describe variations on the standard technique.

As used herein the terms “treat” or “treating” are used interchangeably to include abrogating, substantially inhibiting, slowing or reversing the effects of radiation exposure, substantially ameliorating clinical or aesthetical symptoms of effects of radiation exposure, substantially preventing the appearance of clinical or aesthetical symptoms of effects of radiation exposure, and protecting from effects of radiation exposure. Treating further refers to accomplishing one or more of the following: (a) reducing the severity of the symptoms; (b) limiting development of symptoms; and (c) limiting worsening of symptoms.

Individuals exposed to radiation in the range of 1.0 to 8.0 Gy develop symptoms that reflect injury to the hematopoietic (bone marrow) system. With radiation exposure of 6.0 to 8.0 Gy, a gastrointestinal syndrome develops which is superimposed on the hematopoietic syndrome. Supportive and comfort care is usually indicated for individuals exposed to >10 Gy, since their prognosis is grave. Individuals receiving such high doses are usually killed or severely injured by the blast and thermal effects of a nuclear detonation, although doses in this range could result from accidental or deliberate exposure within a reactor facility or fuel reprocessing plant. Table 1 (adapted from Department of Homeland Security Working Group on Radiological Dispersal Device Preparedness: Medical Preparedness and response Sub-Group (5/1/03 Version)) shows expected clinical symptoms of various levels of radiation exposure.

TABLE 1
Acute Radiation Syndrome

| Radiation | | |
|---|---|---|
| Dose (Gy) | Clinical Status | Clinical Symptoms/Signs |
| 0-1 | Generally | WBC normal or slightly depressed 3-5 weeks |
| | Asymptomatic | following exposure |
| 1-8 | Hematopoietic | Nausea/vomiting, and possibly skin |
| | Syndrome | erythema, fever, mucositis, and diarrhea. |
| | | With whole-body exposure >2 Gy, |
| | | pancytopenia typically occurs 20-30 days |
| | | post exposure. |
| | | Complications may include anemia, immune |
| | | dysfunction, impaired wound healing, |
| | | infections, sepsis, and hemorrhage. |
| 8-30 | Gastrointestinal | Severe nausea/vomiting, watery diarrhea, |
| | Syndrome | often within hours of exposure. In |
| | | addition to the hematopoietic syndrome, |
| | | in severe cases, renal failure and |
| | | vascular collapse. Death from GI |
| | | syndrome may occur within 8-14 days. |
| >20 | Neurovascular | Nausea/vomiting within the first hour |
| | Syndrome | following exposure, prostration, and |
| | | neurological signs of ataxia and confusion. |
| | | Death is inevitable and usually occurs |
| | | within 1-2 days. |

According to the invention, such individuals exposed to radiation are treated by administration of autologous or allogeneic VSELs compositions of the invention. Some subjects will benefit more than others. For example, one or more VSEL stem cell infusion may be used to treat a subject with a radiation exposure dose of 0.1 Gy to 1 Gy, from 1 Gy to 2 Gy, from 2 Gy to 4 Gy, from 4 Gy to 10 Gy, from 10 Gy to 15 Gy, from 15 Gy to 20 Gy, from 20 Gy to 25 Gy, from 30 Gy, or higher. In certain embodiments, a VSEL composition is administered to a subject who develops neutropenia, for example within 8-12 days after exposure. In certain embodiments, a VSEL composition is administered to subject who does not demonstrate hematopoietic recovery within 1-2 weeks following the onset of aplasia (days 25-40 post-exposure). In contrast to treatment with allogeneic HSCs, the VSEL stem cell therapy of the instant invention treats or prevents hematopoietic syndrome, as well as other clinical conditions that would benefit from pluripotent stem cells, including, but not limited to, gastrointestinal and neurological conditions.

The invention further provides methods for administering VSELs in conjunction with disease-related radiation or chemotherapy treatments. More particularly, the invention is used in conjunction with therapies that weaken or suppress the recipient's hematopoietic and/or lymphopoeitic systems. For example, the radiation resistance of VSELs allows for mobilization, collection (with optional expansion and differentiation) and administration of autologous VSELs to a patient having reduced hematopoietic and/or lymphopoeitic function resulting from prior radiation treatment. Thus, without collection and storage of hematopoietic stem cells prior to a radiation or chemotherapy treatment, the patient's hematopoietic and/or lymphopoeitic systems can nevertheless be regenerated or restored.

Beneficial radiation resistance of human VSELs can also be employed in myeloablative and myeloreductive therapies. For example, a subject that will receive a bone marrow transplantation (BMT) typically undergoes a series of pre-treatments that are designed to prepare the bone marrow space to receive administered cells. These pre-treatments can include, but are not limited to treatments designed to suppress the recipient's immune system so that the transplant will not be rejected if the donor and recipient are not histocompatible. An exemplary space-creating pre-treatment comprises exposure to chemotherapeutics that destroy all or some of the bone marrow and total body irradiation (TBI).

Total doses of total body irradiation used in bone marrow transplantation are high, typically up to about 1.5 Gy, and sometimes may be as high as from 10 to >12 Gy. As mentioned, a dose of 3.5 to 4.5 Gy is fatal, 50% of exposed individuals dying within 60 days without aggressive medical care. Total body irradiation both destroys the patient's bone marrow (allowing donor marrow to engraft) and kills residual cancer cells. Such high total body doses are made possible by spreading the total dose out between several sessions, or “fractions,” with an interval of time in between allowing other normal tissues some time to repair some of the damage caused. Fractionated total body irradiation results in lower toxicity and better outcomes than delivering a single, large dose. For example, a fractionated total-body irradiation (FTBI) regimen involving 1320 cGy might consist of 11 fractions of 120 cGy.

The radiation resistance of human VSELs enables treatment methods wherein administration of VSELs can be interspersed or overlap with steps of fractional TBI for myeloablative or myeloreductive therapy. According to the invention, VSEL transplantations can be performed prior to, during the course of, or after completion of steps of myoablative/myeloreductive therapy, such as fractionated total body irradiation. Thus, the invention provides in some embodiments a method wherein a subject has undergone, or will undergo a treatment to at least partially reduce the bone marrow in the subject.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject. For example, suitable formulations can include aqueous and nonaqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and nonaqueous solutions can be used.

The therapeutic methods and compositions of the presently disclosed subject matter can be used with additional adjuvants or biological response modifiers including, but not limited to, cytokines and other immunomodulating compounds.

Suitable methods for administration the compositions of the presently disclosed subject matter include, but are not limited to, intravenous administration and delivery directly to the target tissue or organ (e.g., the bone marrow). In some embodiments, the method of administration encompasses features for regionalized delivery or accumulation of the cells at a target site. In some embodiments, the cells are delivered directly into the target site. In some embodiments, selective delivery of the cells of the presently disclosed subject matter is accomplished by intravenous injection of cells, where they home to the target site and engraft therein.

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated).

In an embodiment of the invention, VSELs (including expanded VSELs and/or VSELs differentiated toward hematopoietic/lymphopoietic cells) are administered once to a subject. In another embodiment, the VSELs are administered to a subject in two or more separate administrations. In an embodiment of the invention, the VSELs are administered in an amount between about 1×10⁴ and 1×10⁵ isolated cells. In another embodiment, the amount of VSELs administered is between about 1×10⁵ and 1×10⁶ isolated VSELs. In another embodiment, the amount of VSELs administered is between about 1×10⁶ and 1×10⁷ isolated VSELs. In another embodiment, the amount of VSELs administered is between about 1×10⁷ and 1×10⁸ isolated VSELs. In further embodiments, VSELs are administered to a subject in amounts between 1×10⁶ and about 2×10⁶, between 2×10⁶ and 5×10⁶, between 5×10⁶ and 1×10⁷, between 1×10⁷ and 2×10⁷, between 2×10⁷ and 5×10⁷, between 5×10⁷ and 1×10⁸, between 1×10⁸ and 2×10⁸, between 2×10⁸ and 5×10⁸, between 5×10⁸ and 1×10⁹. The VSELs may be administered by one route or by more than one route (e.g., bone marrow injection and intravenous). The VSELs may be introduced into the subject at one or more locations (e.g., bone marrow injection at more than one site).

In certain embodiments, the VSELs are provided in a composition that comprises other nucleated cells. In certain such embodiments, the cells of the composition are at least 50% VSELs. In other embodiments, at least 70% of the cells of the composition are VSELs. In other embodiments, at least 80% of the cells of the composition are VSELs or VSELs. In additional embodiments, at least 90% or at least 95% of the cells of the composition are VSELs. In certain embodiments, the cells of the composition are at least 50%, at least 70%, at least 80%, at least 90%, or at least 95% VSELs and cells expanded and/or differentiated from VSELs as described above.

Actual dosage levels of VSELs in the compositions of the presently disclosed subject matter can be varied so as to administer an amount that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

In certain embodiments, in addition to collection and administration of VSELs, radioprotectants, chelating agents, and drugs to enhance radioisotope excretion may also be administered. Radioprotectants (e.g., amifostine, potassium iodide, 5-androstenediol), chelating agents (e.g., DPTA), and drugs which enhance the excretion of radioactive isotopes (e.g., prussian blue, sodium bicarbonate) are important countermeasures for the treatment of subjects exposed to ionizing radiation. Physical barriers can be employed as well. For example, devices that shield head and/or torso may be employed to reduce gastrointestinal, neurovascular, and pulmonary radiation injury such as radiation pneumonitis.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

The VSELs or VSEL-derived compositions can be administered in one or more steps. For example, the whole expanded population can be administered at once. Alternatively, part of the expanded population is administered, and the other part reserved and expanded further, thus providing an initial dose and subsequent booster doses. In certain embodiments of the invention, hVSELs are isolated and prepared and a first dose is administered to the subject within 24 hours after VSEL collection. Further doses are optionally administered as more hVSELs and/or hVSEL-derived cells are prepared.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Irradiated VSELs Rescue Hematopoietic System of Irradiated Mice

Twenty adult male or female transgenic GFP C57BL/6 mice (4-8 weeks old; Jackson Labs) are exposed to a lethal dose (950 cGy) of irradiation. This extreme level of radiation assures that any viable stem cells isolated are only VSELs.

Four days after irradiation, the mice are sacrificed and VSELs isolated from BM by FACS (see, Ratajczak et al., 2011, Exp. Hematol. 39:225). The murine GFP-VSELs are then in subjected to expansion (culturing in methylcellulose plates) and priming (co-culturing over OP9 cells) over a 5-10 day period as described in Ratajczak et al., 2011. The OP9-primed VSELs are isolated by FACS and administered (10⁵ cells) either by tail vein injection or intrafemural administration to C57Bl6 mice (20 mice per group), 24 hrs after being subjected to sublethal (250 cGy) or lethal whole body irradiation (950 cGy). As controls, separate groups of mice (20 per group): i) receive no cell therapy (they should die well before the end of the study period); ii) are treated with non-irradiated GFP-HSCs (10⁵ cells) as described in Ratajczak et al., 2011, which allows for full survival and chimerism of the immune system; or iii) are treated with an equal number of fresh GFP-VSELs, which do not promote survival of the animals. The HSCs and fresh VSELs are administered intra-femorally.

Animals are sacrificed 3 months after initial transplant to measure effects on survival and stimulation of the immune system (white cell count) and for increasing platelet levels. BM and peripheral blood are collected and subjected to FACS and GFP cells stained for hematopoietic and lymphopoietic markers (anti-CD45), anti-CD45R/B220, anti-Gr-1, anti-T cell receptor-αβ, anti-T-cell receptor-γδ, anti-CD11b, anti-Ter119, and anti-Ly-6A/E (Sca-1). These studies test for host-transplant chimerism.

The results show that irradiated VSELs, collected from irradiated donor mice and primed to hematopoietic lineage significantly increase the survival time of sub-lethal and lethally irradiated mice compared to mice not treated, and significantly increase levels of white cells and platelets over a three month period.

### Example 2

### Human VSELs Rescue Hematopoietic System of Lethally Irradiated Mice

Resistance of hVSELs to X-irradiation in vitro. Healthy volunteers are treated with G-CSF (480 μg) two consecutive days to mobilize the VSELs from the BM to peripheral blood and blood (200-300 ml) is collected. To isolate hVSELs, following G-CSF treatment, total nucleated cells are collected by apheresis, and subjected to size-based separation and FACS. The cells are subject to multiple analyses including RT-PCR and fluorescence labeling. RT-PCR is used to analyze expression of Oct4, Nanog, Nkx2.5/Csx, VE-cadherin, and GFAP mRNA levels. Fluorescent staining of hVSELs is used to measure expression of CXCR4, lin, CD45, SSEA-4, Oct-4 and Nanog.

The hVSELs are cultured in vitro and exposed to different doses of X-irradiation. Cell viability is assessed by almarBlue staining, cell counting and in BrdU labeling studies to measure proliferation. Viable cells are tested to determine whether they can be directed to hematopoietic lineage and found to contain CFU-M (colony forming unit-macrophage, CFU-G (colony forming unit-granulocyte) and BFU-E (burst forming unit-erythrocyte).

hVSELs are tested for the ability to rescue mice exposed to whole body irradiation (950 cGy). Lethally irradiated mice essentially have no immune system and therefore do not reject the human cells as foreign. Where rejection is encountered, NOG (NOD/SCIG/IL-2Rgamma deficient) mice are used. It has been shown that human CD34⁺ HSCs can reconstitute the immune system of these mice.

hVSELs are expanded on methylcellulose and co-cultured with OP9 cells or MSCs to prime the hVSELs to hematopoietic lineage. Alternatively, hVSELs are cultured in serum-free medium with stem cell factor (SCF), thrombopoietin (TPO), fibroblast growth factor (bFGF), and FLT3 ligand (Flt3L). 10⁶ hVSELs are administered to separate groups of mice (20 mice per group), via tail vein or intrafemural transplant 24 hours after lethal whole body irradiation, and the effects of the transplant on animal survival and rescue of the immune system is compared to animals receiving no transplant. Ranges of primed hVSELs (e.g., 10³-10⁶ cells) are tested for efficacy as well as multiple administrations and varying doses at different time points (e.g., 10³ cells followed a month later by 10³ cells). Increased doses over time can represent a treatment regimen where fewer autologous VSELs are available at the start of treatment, and greater numbers become available at later times after in vitro expansion. The treatments are evaluated for prolonging survival of the sublethal and lethally irradiated mice, and formation of human-murine chimerism is determined. All major hematopoietic lineages in PB, BM, and spleen can be identified by FACS, using human antigen-specific Abs. The presence of human CD45⁺, CD33⁺, Glycophorin A⁺, CD41a⁺, CD19⁺, CD3⁺, CD133⁺ and CD34⁺ cells can be determined by mAb staining and flow cytometric analysis. Each analysis can be paired with a corresponding matched-isotype control. The antibodies used to detect human cells are not cross-reactive with murine cells.

hVSELs are also tested for the ability to reconstitute a functioning immune system in irradiated mice, capable of responding to immunogens and protecting against infection. Irradiated mice treated with primed hVSELs and surviving for 3 months are challenged with lipopolysaccharide (LPS) which causes a rapid secretion of pro-inflammatory cytokines such as TNF-α, IL-1, IL-6, IL-8 and IFN-γ, and concomitant induction of potent anti-inflammatory factors secreted by monocytes/macrophages such as IL-10 and TGF-β in mice with functioning immune systems.

The results show that lethally irradiated mice that have been treated with primed hVSELs demonstrate increase survival versus non-treated mice and develop a functioning immune system which is responsive to challenge by LPS and other agents.

### Example 3

### Irradiated VSELs Rescue Lethally Irradiated Mice

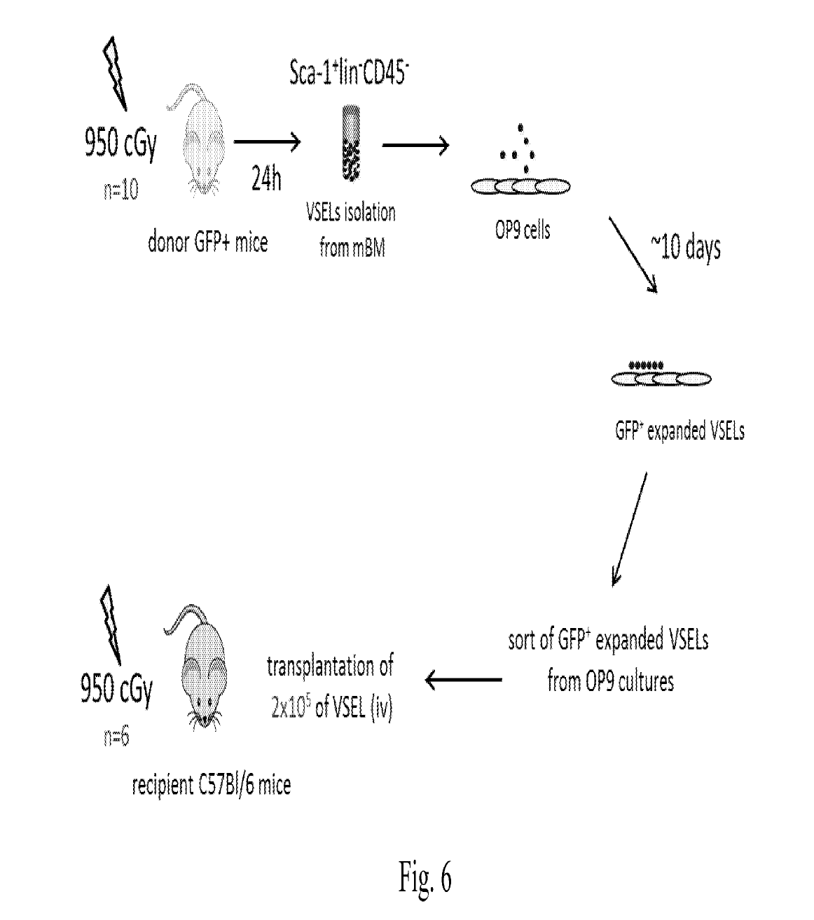

Ten adult male or female transgenic GFP C57BL/6 mice were exposed to a lethal dose (950 cGy) of irradiation. This extreme level of radiation assured that any viable stem cells isolated are only VSELs. The mice were sacrificed and VSELs (Sca-1⁺lin⁻CD45⁻) were isolated from BM by FACS. The murine GFP-VSELs were then co-cultured over OP9 cells for about 10 days. GFP-expressing donor cells were sorted by FACS and administered by tail vein injection to lethally irradiated recipient C57Bl/6 mice. In a first experiment, recipient mice received 1×10⁵ donor VSELs. All of the recipients died approximately 12 days post transplantation. The experiment was repeated using greater amounts of VSELs. In Experiment 2 (FIG. 6) and Experiment 3, recipient mice received 2×10⁵ donor VSELs. In each of the second and third experiments, five of six recipients survived and became chimeric, while the sixth recipient died around 12 days post transplantation.

In each of these experiments, only Sca-1⁺lin⁻CD45⁻ VSELs were able to expand and become specified into the hematopoietic lineage. No hematopoietic activity such as colony formation in vitro or expansion over OP9 cells was observed among Sca-1⁺lin⁻CD45⁺ hematopoietic stem/progenitor cells.

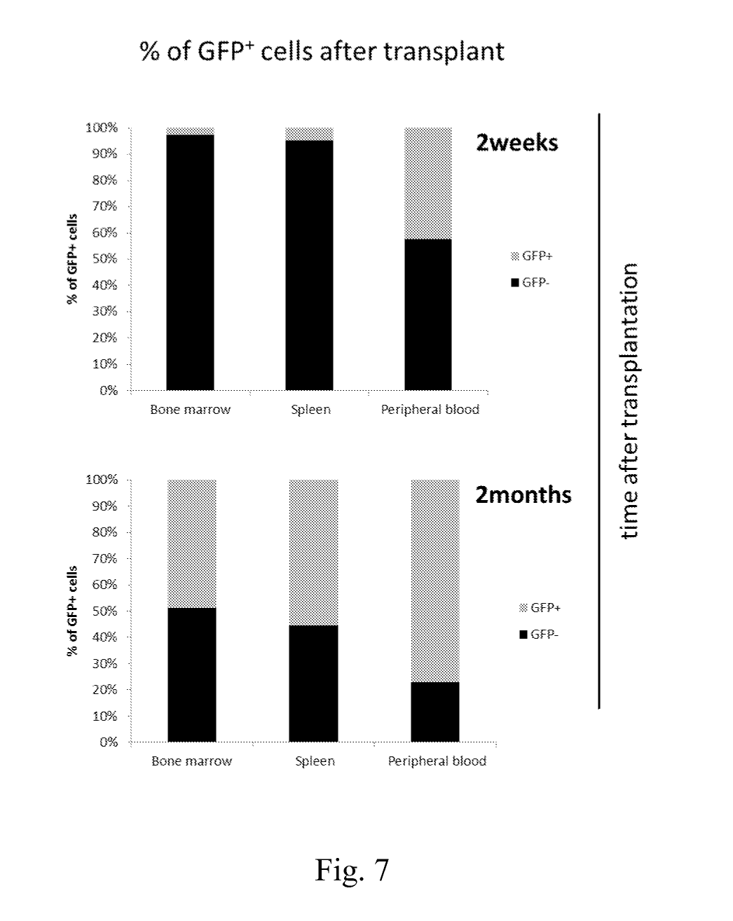

Two weeks after transplantation, GFP⁺ cells from donor VSELs were found mostly in peripheral blood where they constituted nearly half of hematopoietic cells. After two months, GFP⁺ cells constituted about 50% or more of recipient bone marrow and spleen cells. (FIG. 7).

1. A method of treating radiation exposure in a subject, comprising administering a therapeutically effective amount of human very small embryonic-like stem cells (VSELs) to the subject.

2. The method of claim 1, wherein the VSELs are collected from the subject after the radiation exposure.

3. The method of claim 1, wherein the VSELs are collected from the subject prior to the radiation exposure and administered to the subject after the radiation exposure.

4. The method of claim 1, wherein the VSELs are prophylactically administered to the subject prior to or during the radiation exposure.

5. The method of claim 1, wherein the radiation exposure comprises a dose selected from the group consisting of from 0 to 1 Gy, from 1 to 8 Gy, and from 1 to 30 Gy.

6. (canceled)

7. (canceled)

8. The method of claim 1, wherein the radiation exposure comprises a dose greater than 20 Gy.

9. The method of claim 1, wherein the radiation exposure is sufficient to induce acute radiation syndrome in the subject.

10. The method of claim 1, wherein the radiation exposure is sufficient to induce hematopoietic syndrome in the subject.

11. The method of claim 1, wherein the radiation exposure sufficient to induce gastrointestinal syndrome in the subject.

12. The method of claim 1, wherein the radiation exposure is sufficient to induce neurovascular syndrome in the subject.

13. The method of claim 1, wherein the VSELs are autologous to the subject.

14. The method of claim 1, wherein the VSELs are allogeneic to the subject.

15. The method of claim 1, wherein the VSELs are expanded ex vivo prior to administration to the subject.

16. The method of claim 1, wherein the VSELs are primed towards hematopoietic/lymphopoietic differentiation ex vivo prior to administration to the subject.

17. The method of claim 16, wherein the VSELs are primed by culture in serum-free medium with one or more of stem cell factor (SCF), thrombopoietin (TPO), and Flt3 ligand.

18. The method of claim 16, wherein the VSELs are primed by co-culture with bone marrow-derived mesenchymal stem cells (MSCs) or OP9 cells.

19. The method of claim 1, wherein the VSELs comprise CD45⁻/lin⁻/CD34⁺ cells, CD45⁻/lin⁻/CD133⁺ cells, CD45⁻/lin⁻/CD34⁺/CD133⁺/CXCR4⁺ cells, or CD45⁻/GlyA⁻/CD133⁺ cells.

20. The method of claim 1, wherein the VSELs express one or more of Oct-4, Nanog, and SSEA-4.

21. The method of claim 1, wherein the subject is a human.
